(19) United States
(12) Patent Application Publication
(10) Pub. No.: US 2026/0334967 A1
(43) Pub. Date: Oct. 29, 2026

(54) LITHOTRIPSY MEDICAL DEVICE

(71) Applicant: Boston Scientific Scimed, Inc., Maple Grove, MN (US)

(72) Inventors: Javier Palomar-Moreno, Galway (IE); Olena Pernatiy, Galway (IE); Anthony Malone, Galway (IE)

(73) Assignee: Boston Scientific Scimed, Inc., Maple Grove, MN (US)

(21) Appl. No.: 19/658,034

(22) Filed: Apr. 24, 2026

Related U.S. Application Data
(60) Provisional application No. 63/794,635, filed on Apr. 25, 2025.

(51) Int. Cl. A61B 17/22

(52) U.S. Cl. CPC A61B 17/2202; A61B 2017/22027; A61B 2017/22054; A61B 2017/22069

(57) ABSTRACT

Example medical devices are disclosed. A lithotripsy device includes an outer shaft having a distal end region and a lumen, an inner shaft extending within a portion the outer shaft, the inner shaft having a distal end region. The lithotripsy device also includes a balloon having a proximal end attached to the distal end region of the outer shaft, a distal end attached to the distal end region of the inner shaft, and a plurality of inflation lobes positioned between the proximal and distal end regions. The lithotripsy device includes a first force generator coupled to the inner shaft and a first scoring element having a first end attached to the distal end region of the outer shaft and a second end attached to the distal end region of the inner shaft. A portion of the scoring element is free from contact with an outer surface of the balloon.

### CROSS-REFERENCE TO RELATED APPLICATIONS

This application claims the benefit of priority under 35 U.S.C. §119 of U.S. Provisional Application No. 63/794,635, filed Apr. 25, 2025, the entire disclosure of which is hereby incorporated by reference.

### TECHNICAL FIELD

The present disclosure pertains to medical devices, and methods for manufacturing medical devices. More particularly, the present disclosure pertains to lithotripsy devices and methods.

### BACKGROUND

A wide variety of intracorporeal medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example lithotripsy medical device includes an outer shaft having a distal end region and a lumen, an inner shaft extending within at least a portion of the lumen of the outer shaft, the inner shaft having a distal end region. Further, the lithotripsy device also includes a balloon having a proximal end attached to the distal end region of the outer shaft, a distal end attached to the distal end region of the inner shaft, and a plurality of inflation lobes positioned between the proximal end region and the distal end region. Further, the lithotripsy device includes a first force generator coupled to the inner shaft and a first scoring element having a first end attached to the distal end region of the outer shaft and a second end attached to the distal end region of the inner shaft. Further, at least a portion of the scoring element is free from contact with an outer surface of the balloon.

Alternatively or additionally to any of the embodiments above, wherein the first force generator is configured to generate a force wave within a portion of the balloon

Alternatively or additionally to any of the embodiments above, wherein the force wave is configured to transmit mechanical energy to a target region.

Alternatively or additionally to any of the embodiments above, wherein the first scoring element includes a first scoring wire, and wherein the force wave is configured to vibrate the first scoring wire, and wherein the vibration of the first scoring wire scores the target region.

Alternatively or additionally to any of the embodiments above, wherein the mechanical energy from the force wave contacts the target region over a time period, and wherein the scoring wire scores the target region over the time period during which the force wave contacts the target region.

Alternatively or additionally to any of the embodiments above, wherein the force wave is transmitted at a frequency between 70 kilohertz and 100 kilohertz.

Alternatively or additionally to any of the embodiments above, wherein the first force generator includes a ceramic material.

Alternatively or additionally to any of the embodiments above, wherein the first force generator further includes an electrode attached to an outer surface of the ceramic material.

Alternatively or additionally to any of the embodiments above, wherein the balloon includes a first inflation lobe and a second inflation lobe, and wherein second inflation lobe is positioned distal to the first inflation lobe, and wherein a valley is positioned between the first inflation lobe and the second inflation lobe.

Alternatively or additionally to any of the embodiments above, wherein the first inflation lobe and the second inflation lobe are configured to be positioned on either side of a target region in an inflated configuration.

Alternatively or additionally to any of the embodiments above, further comprising a second scoring element having a first end attached to the distal end region of the outer shaft and a second end attached to the distal end region of the inner shaft.

Alternatively or additionally to any of the embodiments above, wherein the balloon is designed to shift between an unexpanded configuration and an expanded configuration wherein the balloon is in contact with a target region, and wherein the first scoring element is positioned between the outer surface of the balloon and the target region in the expanded configuration.

A lithotripsy medical device system includes a console, an outer shaft having a distal end region and a lumen, an inner shaft extending within at least a portion of the lumen of the outer shaft, the inner shaft having a distal end region. Further, the lithotripsy device also includes a balloon having a proximal end attached to the distal end region of the outer shaft, a distal end attached to the distal end region of the inner shaft, and a plurality of inflation lobes positioned between the proximal end region and the distal end region. Further, the lithotripsy device also includes a force generator coupled to the inner shaft and the console and a scoring element having a first end attached to the distal end region of the outer shaft and a second end attached to the distal end region of the inner shaft. Further, at least a portion of the scoring element is free from contact with an outer surface of the balloon.

Alternatively or additionally to any of the embodiments above, wherein the force generator is configured to generate a force wave within a portion of the balloon.

Alternatively or additionally to any of the embodiments above, wherein the force wave is configured to transmit mechanical energy to a target region.

Alternatively or additionally to any of the embodiments above, wherein the first scoring element includes a first scoring wire, and wherein the force wave is configured to vibrate the scoring wire, and wherein the vibration of the scoring wire scores the target region.

Alternatively or additionally to any of the embodiments above, wherein the mechanical energy from the force wave contacts the target region over a time period, and wherein the scoring wire scores the target region over the time period during which the force wave contacts the target region.

Alternatively or additionally to any of the embodiments above, wherein the force wave is transmitted at a frequency between 70 kilohertz and 100 kilohertz.

Alternatively or additionally to any of the embodiments above, wherein the balloon is designed to shift between an unexpanded configuration and an expanded configuration wherein the balloon is in contact with the target region, and wherein the scoring wire is positioned between the outer surface of the balloon and the target region in the expanded configuration.

A method for treating a body vessel includes disposing a medical device within the body vessel at a position adjacent to a lesion. The medical device includes an outer shaft having a distal end region and a lumen, an inner shaft extending within at least a portion of the lumen of the outer shaft, the inner shaft having a distal end region. The medical device also includes a balloon having a proximal end attached to the distal end region of the outer shaft, a distal end attached to the distal end region of the inner shaft, and a plurality of inflation lobes positioned between the proximal end region and the distal end region. The medical device also includes a force generator coupled to the inner shaft and a scoring element having a first end attached to the distal end region of the outer shaft and a second end attached to the distal end region of the inner shaft, wherein at least a portion of the scoring element is free from contact with an outer surface of the balloon. The method also includes generating a force wave with the force generator over a time period and vibrating the scoring wire over the time period, and wherein the force wave and the scoring element at least partially break apart the lesion.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example medical device;

FIG. 2 is a perspective view of the medical device of FIG. 1 in a first configuration;

FIG. 3 is a perspective view of the medical device of FIG. 1 in a second configuration;

FIG. 4 is a perspective view of a portion of the medical device of FIG. 1;

FIG. 5 is a cross-sectional view taken along line 5-5 of FIG. 4;

FIGS. 6-8 illustrate the medical device of FIG. 1 being used to treat a target region within a body vessel.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

The use of force generating medical devices may be a desirable method for treating intravascular lesions in a blood vessel. In some instances, calcification along or otherwise adjacent to the vessel wall can complicate an intervention. Disclosed herein are force generating medical devices and methods that are designed to improve the treatment of intravascular lesions. The devices and methods disclosed herein may be described as force generating medical devices/methods in that the devices may generate and transfer a force to the treatment area to break up the lesion.

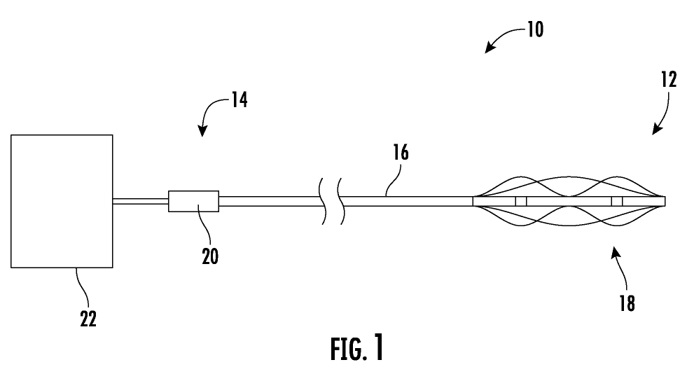

FIG. 1 illustrates an example medical device 10 having a distal end region 12 and a proximal end region 14. The medical device 10 may include an outer shaft 16. The outer shaft 16 may include one or more lumens extending therein. For example, the outer shaft 16 may include an inflation lumen and a guidewire lumen. Additionally, the medical device 10 may include a force generating assembly 18 positioned adjacent a distal end of the outer shaft 16.

FIG. 1 further illustrates that the medical device 10 may further include a controller 20 coupled to a proximal end of the catheter shaft 16. The controller 20 may include one or more actuators (e.g., buttons, selectors, etc.) which permit a clinician to operate one or more components of the force generating assembly 18 of the medical device 10. Additionally, FIG. 1 illustrates that the controller 20 may be connected to a console 22 (e.g., generator, energy generator, electrical generator). As will be described in greater detail below, the console 22 may be configured to send electrical signals along electrical leads or electrical wires disposed within the catheter shaft 16 to one or more force generators positioned within the force generating assembly 18. The force generators may convert the electrical energy received from the console 22 to force waves which act upon a target site adjacent to the force generating assembly 18.

For purposes of this disclosure, a lithotripsy medical device may be understood to be a device designed to generate and/or transmit forces to a target region in a manner that may break up the target region. In at least some instances, the generation of forces may occur in a repeated manner with waves of forces that are configured to impact the target region. The force generation could occur in a regular manner with equally spaced time intervals between force generations, or the force generations could occur with differing time intervals between force generations.

As discussed herein, a variety of the different generators may be used to generate and transmit forces within the balloon 68. For example, the force generators disclosed herein may include mechanical wave generators, pressure wave generators, pressure wave transducers, acoustic transducers, ultrasound transducers, ultrasound generators, electrical arc generators, electrical emitters, electrical arc generators, sound-wave generators (including generation of sound waves propagating equal to or slower than the speed of sound), shock-wave generators (including generation of shock waves propagating faster than the speed of sound), sonic-wave generators, plasma generators, corona discharge generators, cavitation bubble generators, or any other similar or suitable force generator. Any of the example force generators disclosed herein may be used alone or in combination with any other force generator to generate and transmit a variety forces to the target site.

In other examples, the force generators disclosed herein may include a corona generator which is configured to receive pulsed electrical signals sent from a power pulse generator, whereby under the effect of the pulsed electrical signals, an electrolyte inflation media may undergo a corona reaction, causing molecular ionization and vaporization of the electrolyte inflation media. The vapor bubbles produced by the molecular ionization may squeeze the electrolyte liquid, thereby rapidly increasing the pressure and force generation inside the balloon 68.

In other examples, the force generators disclosed herein may include one or more emitters (e.g., fiber optic emitters which emit light of any wavelength) which are configured to emit one or more pulses of light energy (e.g., laser light, white light, visible light, infrared light, ultraviolet light, etc.) from the emitter, wherein emitting the light energy may react with the liquid inflation media and generate plurality of propagating force waves directed toward the treatment area to break up a lesion.

Further, any of the example force generators described herein may use any technique to generate force waves. For example, the force generators may use acoustic energy, electrical energy, laser energy, laser-induced force wave, light energy (e.g., laser light, white light, visible light, infrared light, ultraviolet light, etc.), light pulse generation in the inflation media, etc. to generate force waves within the balloon.

Further, the medical device 10 may include one or more components configured to generate an electrical discharge from one or more electrical emitters which vaporize the liquid within the balloon 68, creating a rapidly expanding and collapsing bubble (e.g., a cavitation bubble) that generates force waves (e.g., sonic pressure waves, shock waves). In other examples, the medical device 10 may include one or more components configured to convert laser energy distributed through optical fibers to force waves (e.g., acoustic pressure waves) by generating a plasma event within the inflation media used to inflate the balloon 68. Additionally, the medical device 10 may further generate a secondary cavitation bubble which is configured to transmit additional force waves toward the target region.

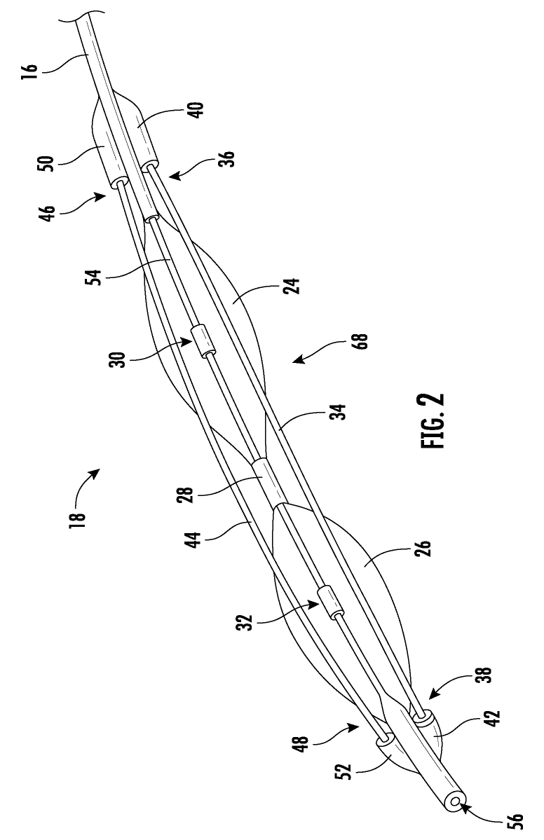

FIG. 2 is a perspective view of the medical device of FIG. 1 in a first configuration illustrating the force generating assembly 18. As discussed herein, the force generating assembly 18 may be positioned adjacent to the distal end region of the outer shaft 16. FIG. 2 illustrates that the force generating assembly 18 may include an inner shaft 54 extending through a lumen of the outer shaft 16. Further, the inner shaft 54 may include a lumen 56 extending therein. It can be appreciated that the lumen 56 of the inner shaft 54 may be configured to permit a guidewire to extend therethrough. Further, the inner shaft 54 may extend the entire length of the outer shaft 16. Further yet, the inner shaft 54 may extend through the controller 20, thereby permitting a guidewire to extend from a position outside the controller 20 and through the entire length of the inner shaft 54.

FIG. 2 illustrates that the force generating assembly 18 may also include a balloon 68 including a first inflation lobe 24 and a second inflation lobe 26, whereby the first lobe 24 is in fluid communication with the second lobe 26. The balloon 68 may include a proximal end (e.g., a proximal waist) which is attached to the distal end of the outer shaft 16. Further, the balloon 68 may include a distal end (e.g., a distal waist) which is attached to the distal end of the inner shaft 54. It can be appreciated that the configuration of the force generating assembly 18 shown in FIG. 2 may illustrate a configuration in which the balloon 68 is in a deflated configuration, whereby the first and second lobes 24, 26 of the balloon 68 are in a radially compact, low-profile configuration for tracking the force generating assembly 18 through a body lumen.

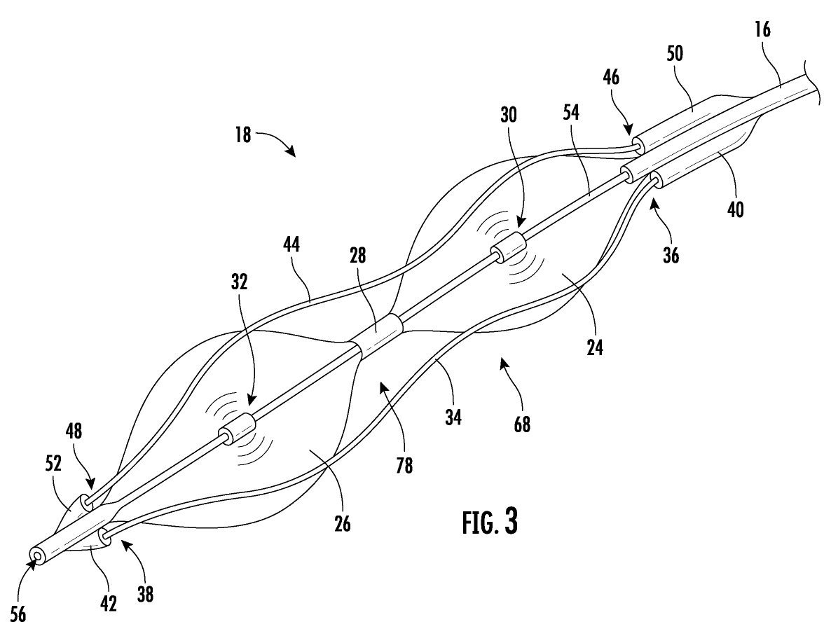

As will be described in greater detail below, in some examples, the force generating assembly 18 may further include a constriction ring 28 positioned between a distal end region of the first lobe 24 and a proximal end region of the second lobe 26. In some examples, the constriction ring 28 may be fixedly attached (e.g., bonded, glued, etc.) to the outer surface of the balloon 68. In other examples, the constriction ring 28 may extend circumferentially around the outer surface of the balloon 68 while also being free to spin, rotate, slide, etc. along the outer surface of the balloon 68. However, it can be appreciated that, in other examples, the force generating assembly 18 may not include a constriction ring 28, yet the balloon 68 may still maintain the general, curvilinear shape illustrated in FIGS. 2-3.

FIG. 2 further illustrates that the force generating assembly 18 may further include a first force generator 30 positioned within an inner cavity of the lobe 24 and second force generator 32 positioned within an inner cavity of the lobe 26. As will be described in greater detail below, each of the first force generator 30 and the second force generator 32 may be configured to generate a force (e.g., force wave, etc.) that may be transmitted through inflation media (used to inflate the balloon 68) toward a target region (e.g., calcified lesion). Along with other components of the force generating assembly 18, the transmitted forces generated by the first force generator 30 and the second force generator 32 may contact and break up the calcified lesion. As discussed herein, the force generators 30, 32 may be configured to convert electrical energy to other forms of energy within inflation media used to inflate the balloon 68.

FIG. 2 further illustrates that the force generating assembly 18 may further include a first scoring element 34 (e.g., scoring wire, scoring filament, scoring coil, scoring thread, scoring cable, etc.) and a second scoring element 44 (e.g., scoring wire, scoring filament, scoring coil, scoring thread, scoring cable, etc.), whereby each of the first scoring element 34 and the second scoring element 44 extend generally long the length of the balloon 68. In some examples, the force generating assembly 18 may include one, two, three, four, or more scoring elements circumferentially positioned around the balloon 68 (e.g., 180 degrees, 120 degrees, or 90 degrees apart).

FIG. 2 further illustrates that the first scoring element 34 may include a proximal end region 36 which is coupled to a first proximal attachment region 40 and a distal end region 38 which is coupled to a first distal attachment region 42. Similarly, FIG. 2 further illustrates that the second scoring element 44 may include a proximal end region 46 which is coupled to a second proximal attachment region 50 and a distal end region 48 which is coupled to a second distal attachment region 52. It can be appreciated that the portion of the first scoring element 34 extending between the proximal end region 36 and the distal end region 38 may be free from attachment to the outer surface of the balloon 68. Similarly, it can be appreciated that the portion of the second scoring element 44 extending between the proximal end region 46 and the distal end region 48 may be free from attachment to the outer surface balloon 68. In other words, it can be appreciated that the portions of the first scoring element 34 which are not coupled to the first proximal attachment region 40 and the first distal attachment region 42 (e.g., portions of the first scoring element 34 which extend along the lobe 24 and the lobe 26) may be unattached to the outer surface of the balloon 68. Similarly, portions of the second scoring element 44 which are not coupled to the second proximal attachment region 50 and the second distal attachment region 52 (e.g., portions of the second scoring element 44 which extend along the lobe 24 and the lobe 26) may be unattached to the outer surface of the balloon 68.

It can be appreciated from FIG. 2 that the first proximal attachment region 40 and the second proximal attachment region 50 may be formed from a portion of the outer shaft 16. It can be further appreciated from FIG. 2 that the first distal attachment region 42 and the second distal attachment region 52 may be formed from a portion of the inner shaft 54.

In some examples, the first proximal attachment region 40 may be configured to include a cavity, opening, aperture, etc. which permits at least a portion of the proximal end region 36 of the first scoring element 34 to be inserted therein. Similarly, the second proximal attachment region 50 may be configured to include a cavity, lumen, aperture, etc. which permits at least a portion of the proximal end region 46 of the second scoring element 44 to be inserted therein. Further, in some examples, the proximal end region 36 of the first scoring element 34 may be configured to be fixedly attached (e.g., fixedly bonded, glued, etc.) within the cavity, opening, aperture, etc. of the first proximal attachment region 40. Additionally, the proximal end region 36 of the first scoring element 34 may be configured to include extra “slack” within the cavity, opening, aperture, etc. of the first proximal attachment region 40, whereby the slack is configured to permit a portion of the first scoring element 34 to extend out of the cavity, opening, aperture, etc. of the first proximal attachment region 40 as the balloon 68 is inflated. Similarly, the proximal end region 46 of the second scoring element 44 may be configured to be fixedly attached (e.g., fixedly bonded, glued, etc.) within the cavity, opening, aperture, etc. of the second proximal attachment region 50. Additionally, the proximal end region 46 of the second scoring element 44 may be configured to include extra “slack” within the cavity, opening, aperture, etc. of the second proximal attachment region 50, whereby the slack is configured to permit a portion of the second scoring element 44 to extend out of the cavity, opening, aperture, etc. of the second proximal attachment region 50 as the balloon 68 is inflated.

FIG. 2 further illustrates that the first distal attachment region 42 may be configured to include a cavity, opening, aperture, etc. which permits the distal end region 38 of the first scoring element 34 to be inserted therein. Similarly, the second distal attachment region 52 may be configured to include a cavity, lumen, aperture, etc. which permits the distal end region 48 of the second scoring element 44 to be inserted therein. Further, in some examples, the distal end region 38 of the first scoring element 34 may be configured to be fixedly attached (e.g., fixedly bonded, glues, etc.) within the cavity, opening, aperture, etc. of the first proximal attachment region 42. Further yet, in some examples, the distal end region 48 of the second scoring element 44 may be configured to be fixedly attached (e.g., fixedly bonded, glues, etc.) within the cavity, opening, aperture, etc. of the second proximal attachment region 52.

FIG. 3 is a perspective view of the medical device of FIG. 1 that illustrates the force generating assembly 18 in a configuration in which the balloon 68 is in an expanded configuration, whereby the first and second lobes 24, 26 of the balloon 68 are in an inflated configuration for transferring a force (e.g., pressure wave, etc.) through inflation media (used to inflate the balloon 68) toward a calcified lesion.

It can be appreciated from the expanded configuration of FIG. 3 that the inflated balloon 68 may include a generally curvilinear shape with the first lobe 24 and the second lobe 26 having similar spherical shapes. However, this is not intended to be limiting. Rather, the first lobe 24 may be shaped different from the second lobe 26. For example, the first lobe 24 may be generally larger in shape than the second lobe 26. In some examples, it may be beneficial to have a generally larger shaped first lobe 24 (relative to the second lobe 26) when the balloon 68 is being used within a tapered vessel. Alternatively, the second lobe 26 may be generally larger in shape than the first lobe 24.

Further, in some examples, each of the first lobe 24 and the second lobe 26 may include a variety of different geometric shapes. For example, the first lobe 24, the second lobe 26 or both the first lobe 24 and the second lobe 26 may be generally conical, elongated conical, elliptical, ovular, square, rectangular, triangular, tapered, dog-bone, stepped, heart-shaped, serrated or any other suitable shape. In some examples, the overall profile of the balloon 68 may include a sinusoidal shape, whereby the profile of the balloon 68 includes an undulating, wave-like shape. Further, FIG. 3 illustrates that when in the expanded configuration, the balloon 68 may include a “valley” region 78 located between the first lobe 24 and the second lobe 26. For purposes of discussion herein, the valley region 78 may include a space, gap, span, open region, etc. extending between the first lobe 24 and the second lobe 26.

Further, it can be appreciated that the balloon 68 may include more or less than two inflation lobes 24, 26. For example, the balloon 68 may include one, two, three, four, five, six or more lobes whereby the lobes are aligned along a longitudinal axis of the inner shaft 54 to define an elongated, multi-lobe balloon. It can be further appreciated that example balloons 68 which include one, two, three, four, five, six or more lobes (whereby the lobes are aligned along a longitudinal axis of the inner shaft 54) may also include one, two, three, four, five, six or more valley regions 78 extending between the lobes. For example, a balloon which includes three lobes may have two valley regions spaced between the three lobes, a balloon which includes four lobes may have three valley regions spaced between the four lobes, etc.

As discussed herein, it can be appreciated that the first lobe 24 may be in fluid communication with the second lobe 26. Accordingly, inflation media may be transported through the lumen of the outer shaft 16 and into the inner inflation cavity of the first lobe 24, whereby the inflation media may further be transported into from the inflation cavity of the first lobe 24 into the inflation cavity of the second lobe 26. In other words, a single injection of inflation media may inflate both the first lobe 24 and the second lobe 26 (or additional lobes) of the balloon 68. Further, in some examples, one or more lobes of the balloon 68 may be independently inflatable from one or more other lobes. For example, each lobe 24, 26 may be independently inflatable relative to the other lobe 24, 26, respectively.

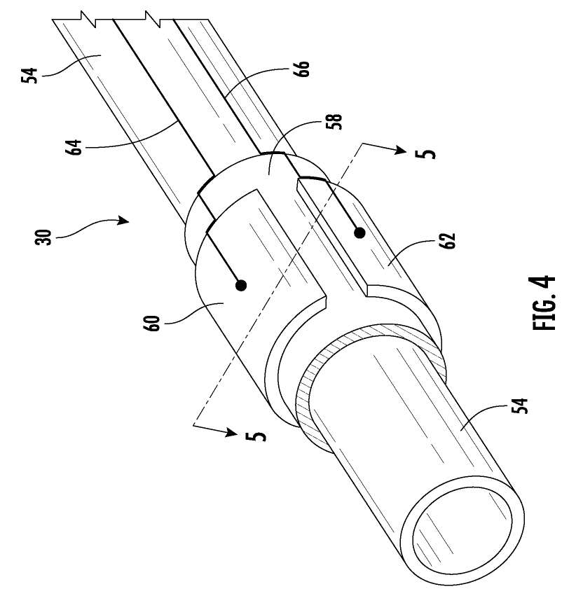

FIG. 4 is a perspective view of a portion of the medical device of FIG. 1 that illustrates an example force generator 30. While FIG. 4 and the following discussion is directed at the force generator 30, it can be appreciated that the force generator 32 illustrated in FIGS. 2-3 may be similar in form and function to the force generator 30 discussed herein. It can be appreciated from FIGS. 2-3 that the force generator 30 may be positioned within the first lobe 24 of the balloon 68 and the force generator 32 may be positioned within the second lobe 26 of the balloon 68.

FIG. 4 illustrates that, in some examples, the force generator 30 may include a ceramic member 58 attached to an outer surface of the inner shaft 54. In some examples, the ceramic member 58 may be fixedly attached (e.g., bonded, glued, etc.) to the outer surface of the inner shaft 54. Additionally, in some examples, the ceramic member 58 may include a piezoelectric ceramic material which may be formed from a plurality of asymmetric crystals configured to deform when activated by an electrical current passing therethrough. For example, the ceramic member 58 may be configured to rapidly expand and contract in a cyclical manner in response to an alternating electrical current being passed through the ceramic material. As will be discussed in greater detail below, the rapid expansion and contraction of the ceramic material may be configured to generate high frequency vibrations within the inflation media utilized to inflate the first and second lobes 24, 26 of the balloon 68. For example, the rapid expansion and contraction of the ceramic material may be configured to generate force waves within the inflation media utilized to inflate the first and second lobes 24, 26 of the balloon 68, whereby the force waves are configured to emanate radially outward from the ceramic member 58 toward a target site (e.g., calcified lesion) adjacent the wall of the balloon 68.

Further, it can be appreciated from FIG. 4 that the force generator 30 may include a first electrode 60 and a second electrode 62 positioned on the ceramic member 58. It can be appreciated that each of the first electrode 60 and the second electrode 62 may be fixedly attached (e.g., bonded, glued, etc.) to the outer surface to the ceramic member 58. Further, FIG. 4 illustrates that the first electrode 60 may be coupled to an electrical lead 64 which may extend to the power supply or console 22 shown in FIG. 1. Further yet, FIG. 4 illustrates that the second electrode 62 may be coupled to an electrical lead 66 which may extend to the power supply or console 22 shown in FIG. 1.

Additionally, in some examples, it can be appreciated that the power supply or console 22 may be configured to generate an alternating current which is transmitted to the ceramic member 58 via, for example, traveling through the electrical lead 64 to the electrode 60, passing through the ceramic member 58 and then traveling back to the power supply or console 22 through the electrode 62 and the electrical lead 66. After reaching a peak magnitude, the electrical signal may change directions, whereby it travels through the electrical lead 66 to the electrode 62, passes through the ceramic member 58 and then travels back to the power supply or console 22 through the electrode 60 and the electrical lead 64. As discussed herein, it can be appreciated that this cyclical, alternating current may cause the rapid expansion and contraction of the ceramic member 58, which, in turn, may generate a series of force waves within the inflation media utilized to inflate the first and second lobes 24, 26 of the balloon 68.

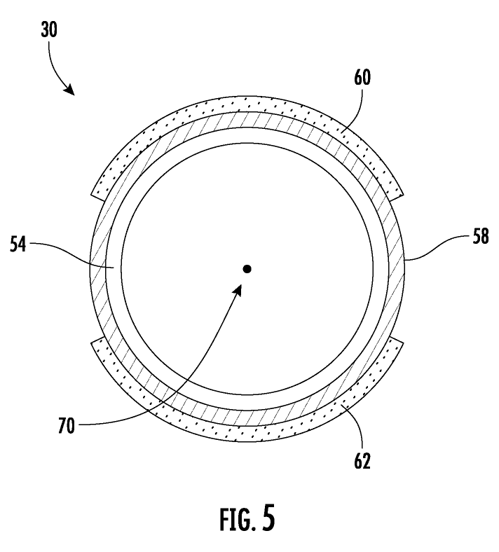

FIG. 5 illustrates a cross-sectional view taken along line 5-5 of FIG. 4. FIG. 5 illustrates the ceramic member 58 attached to an outer surface of the inner shaft 54. It can be appreciated from FIG. 5 that the ceramic member may extend circumferentially around a longitudinal axis 70 of the inner shaft 54. Further, FIG. 5 illustrates the first electrode 60 and the second electrode 62 attached to the outer surface of the ceramic member 58. In some examples, the first electrode 60 may be circumferentially offset from the second electrode 62 by approximately 180 degrees.

It can further be appreciated that the medical device 10 (shown in FIG. 1) may include one or more metallic markers (e.g., fluoroscopic marker bands) to aid with guidance and placement of the medical device 10 during a medical procedure. In some examples, each of the first force generator 30 and the second force generator 32 may include one or more metallic markers (e.g., fluoroscopic marker bands) to aid with guidance and placement of the medical device 10 during a medical procedure.

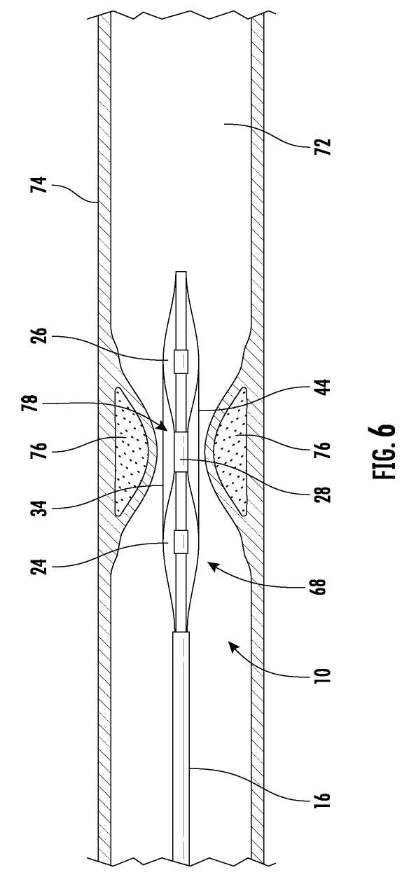

FIGS. 6-8 illustrate the medical device 10 being used to treat a calcified lesion 76 (e.g., a nodular calcification) within a body vessel 74. FIGS. 6-8 describe the medical device 10 being used to treat a calcified lesion 76, however, it can be appreciated that the medical device 10 may be used to treat other types of target regions and/or target tissues.

FIG. 6 further illustrates the medical device 10 having been advanced within a lumen 72 of the body vessel 74 to a position adjacent a calcified lesion 76. It can appreciated from FIG. 6 that, when suitably positioned, the first lobe 24 may be generally positioned on a side of the calcified lesion 76 while the second lobe 26 may be generally positioned on an opposite side of the calcified lesion 76, whereby inflation of the balloon 68 will position the first lobe 24 and the second lobe 26 such that together they “trap” the calcified lesion within the valley region 78 located between the first lobe 24 and the second lobe 26.

FIG. 6 further illustrates the first scoring element 34 and the second scoring element 44 extending along the outer surface of the balloon 68. As discussed herein, it can be appreciated that the portions of the first scoring element 34 and the second scoring element 44 extending along the outer surface of the balloon 68 (including along the valley region 78) may be free to move independently of the first lobe 24 and the second lobe 26 of the balloon 68. For example, as discussed herein, the portions of the first scoring element 34 that are unattached to the first proximal attachment region 40 (shown in FIG. 3) and the first distal attachment region 42 (shown in FIG. 3) and portions of the second scoring element 44 that are unattached to the second proximal attachment region 50 (shown in FIG. 3) and the second distal attachment region 52 (shown in FIG. 3) may be free to move relative to the outer surface of the balloon 68 (including along the valley region 78) defining the first lobe 24 and the second lobe 26.

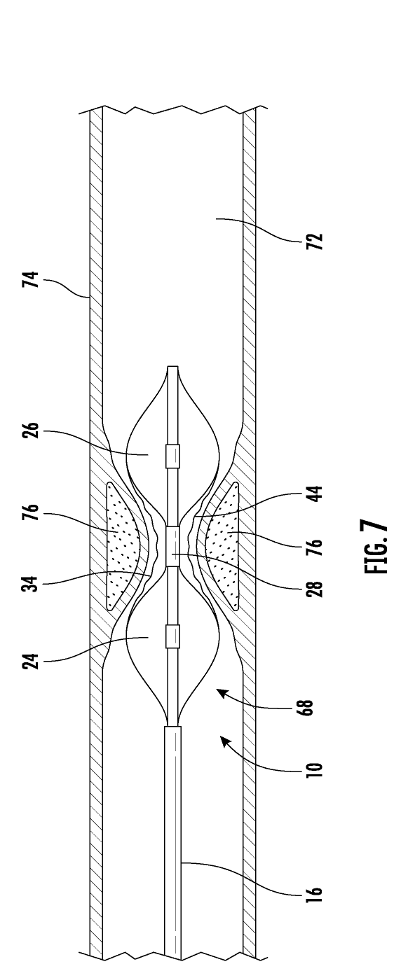

Inflation of the balloon 68 shown in FIG. 6 is schematically depicted in FIG. 7. Inflating the balloon 68 may occur by infusing an inflation media into the balloon 68 (e.g., via an inflation lumen of the catheter shaft 16). In some examples, inflation of the balloon 68 may include inflating the balloon 68 such that the first lobe 24, the second lobe 26 or both the first lobe 24 and the second lobe 26 of the balloon 68 are adjacent, but not contacting the wall of the body vessel 74. In other examples, inflation of the balloon 68 may include inflating the balloon 68 such that the first lobe 24, the second lobe 26 or both the first lobe 24 and the second lobe 26 come into contact with the wall of the body vessel 74. This may include simply contacting the vessel wall or, in some instances, inflating the balloon 68 may include expanding the body vessel 74.

FIG. 7 further illustrates the first scoring element 34 and the second scoring element 44 extending along the outer surface of the balloon 68. As discussed herein, it can be appreciated that the portions of the first scoring element 34 and the second scoring element 44 extending along the outer surface of the balloon 68 (including along the valley region 78) may be free to move independently of the first lobe 24 and the second lobe 26 of the balloon 68.

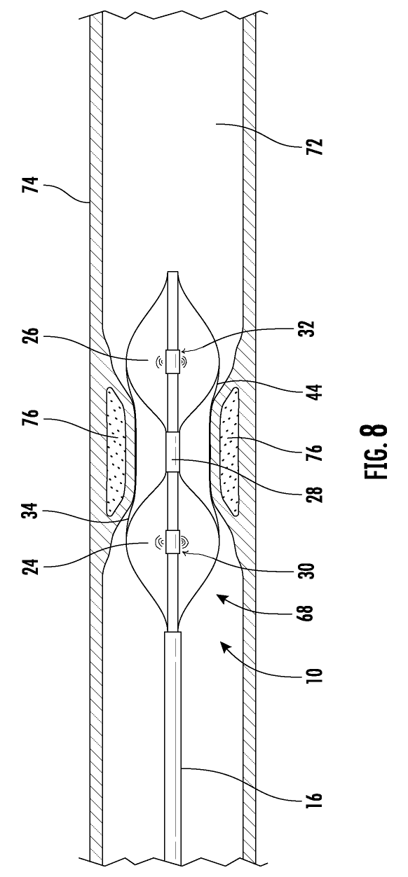

With the balloon 68 inflated (or partially inflated), the first force generator 30 and the second force generator 32 may be activated. For example, FIG. 8 illustrates the inflation (or partial inflation) of the balloon 68 and subsequent activation of the first force generator 30 and the second force generator 32.

FIG. 8 illustrates that the first lobe 24 and the second lobe 26 of the balloon 68 after having been inflated (or partially inflated) with inflation media (e.g., saline, contrast, a saline-contrast mix, etc.). FIG. 8 further illustrates the first scoring element 34 and the second scoring element 44 have been positioned between an outer surface of the balloon 68 and the body vessel 74 such that each of the first scoring element 34 and the second scoring element 44 are contacting the outer surface of the balloon 68 and/or the body vessel 74 adjacent to the calcified lesion 76. As discussed herein, it can be appreciated that even while contacting the wall of the body vessel 74 adjacent the calcified lesion 76, the portions of the first scoring element 34 and the second scoring element 44 extending along the outer surface of the balloon 68 are free to move independently relative to the outer surface of the balloon defining the first lobe 24 and the second lobe 26. In some examples, at least a portion of the first scoring element 34, the second scoring element 44 or both the first scoring element 34 and the second scoring element 44 which extend along the valley region 78 of the balloon 68 may be free of the surface of the balloon 68 when the balloon 68 is inflated and the first force generator 30 and the second force generator 32 are activated to generate force waves within the balloon 68.

Further, as discussed herein, activating the first force generator 30 may create a force wave that emanates away from the force generator 30 and is directed toward the calcified lesion 76. Similarly, activating the second force generator 32 may create a force wave that emanates away from the force generator 32 and is directed toward the calcified lesion 76. Accordingly, FIG. 8 illustrates the activation of first force generator 30 and the second force generator 32 to create force waves directed toward the calcified lesion 76. It can be appreciated that, in some examples, the force waves may include high frequency force waves which impart high frequency forces directed at breaking up the calcified lesion 76. Further, it can be appreciated that the multi-lobe configuration of the balloon 68 may aid in focusing the force wave toward the calcified lesion 76. As discussed herein, the constriction ring 28 may help to maintain that multi-lobe balloon configuration (as shown in FIGS. 3 and 8) when the balloon 68 is inflated. However, in other examples, the balloon 68 may be configured to maintain the curved shaped shown in FIGS. 3 and 8 even when a constriction ring 28 is not positioned on the balloon 68.

Additionally, it can be appreciated that the force waves generated by the first force generator 30 and the second force generator 32 may pass through and/or engage the wall of the balloon 68 such that the first scoring element 34, the second scoring element 44 or both the first scoring element 34 and the second scoring element 44 vibrate along the body vessel 74 at a position adjacent the calcified lesion 76. It can be appreciated that the vibration of the first scoring element 34 and/or the second scoring element 44 may score the body vessel 74 at a position adjacent the calcified lesion 76, thereby enhancing the effect of the force waves to break up the calcified lesion 76. It can be appreciated that the outwardly-directed forces of the force waves may transmit mechanical energy and/or a mechanical forces to the calcified lesion 76. Further, the mechanical forces created by the force waves, together with the vibration (e.g., scoring effect) of the scoring elements 34, 44, may directly or indirectly contact and break up the calcified lesion 76. With the calcified lesion 76 broken up, the balloon 68 may be further inflated to a higher pressure, greater than the initial inflation pressure, to treat the blood vessel 74.

It can be appreciated that for the example medical devices 10 described herein, each first and second force generator 30, 32 may emit a frequency wavelength. The force generators 30, 32 disclosed herein may operate within a wide range of frequency wavelengths. In some examples, the wavelength frequency emitted by each force generator 30, 32 may be in a range of about 10 kilohertz (kHz) to about 110 kHz, or in a range of about 20-90 kHz, or in a range of about 60-100 kHz, or in a range of about 40-80 kHz, or in a range of about 30-60 kHz, or in the range of about 20-50 kHz.

In some examples, the outwardly-directed forces of the force waves may transmit mechanical energy and/or a mechanical forces to the calcified lesion 76 over a time period of about 10 seconds to about 90 seconds, about 20 seconds to about 80 seconds, about 30 seconds to about 60 seconds, about 40 seconds to about 70 seconds, about 30 seconds to about 50 seconds.

The materials that can be used for the various components of the medical device 10 disclosed herein may include those commonly associated with medical devices. The medical device 10 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10 or portions thereof, may be made of a material that does not substantially distort the image and create substantial artifacts (e.g., gaps in the image). Certain ferromagnetic materials, for example, may not be suitable because they may create artifacts in an MRI image. The medical device 10 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A lithotripsy medical device, comprising:
an outer shaft having a distal end region and a lumen;
an inner shaft extending within at least a portion of the lumen of the outer shaft, the inner shaft having a distal end region;
a balloon having a proximal end attached to the distal end region of the outer shaft, a distal end attached to the distal end region of the inner shaft, and a plurality of inflation lobes positioned between the proximal end region and the distal end region;
a first force generator coupled to the inner shaft; and
a first scoring element having a first end attached to the distal end region of the outer shaft and a second end attached to the distal end region of the inner shaft;
wherein at least a portion of the scoring element is free from contact with an outer surface of the balloon.

2. The lithotripsy medical device of claim 1, wherein the first force generator is configured to generate a force wave within a portion of the balloon.

3. The lithotripsy medical device of claim 2, wherein the force wave is configured to transmit mechanical energy to a target region.

4. The lithotripsy medical device of claim 3, wherein the first scoring element includes a first scoring wire, and wherein the force wave is configured to vibrate the first scoring wire, and wherein the vibration of the first scoring wire scores the target region.

5. The lithotripsy medical device of claim 4, wherein the mechanical energy from the force wave contacts the target region over a time period, and wherein the scoring wire scores the target region over the time period during which the force wave contacts the target region.

6. The lithotripsy medical device of claim 5, wherein the force wave is transmitted at a frequency between 70 kilohertz and 100 kilohertz.

7. The lithotripsy medical device of claim 6, wherein the first force generator includes a ceramic material.

8. The lithotripsy medical device of claim 7, wherein the first force generator further includes an electrode attached to an outer surface of the ceramic material.

9. The lithotripsy medical device of claim 1, wherein the balloon includes a first inflation lobe and a second inflation lobe, and wherein second inflation lobe is positioned distal to the first inflation lobe, and wherein a valley is positioned between the first inflation lobe and the second inflation lobe.

10. The lithotripsy medical device of claim 9, wherein the first inflation lobe and the second inflation lobe are configured to be positioned on either side of a target region in an inflated configuration.

11. The lithotripsy medical device of claim 1, further comprising a second scoring element having a first end attached to the distal end region of the outer shaft and a second end attached to the distal end region of the inner shaft.

12. The lithotripsy medical device of claim 1, wherein the balloon is designed to shift between an unexpanded configuration and an expanded configuration wherein the balloon is in contact with a target region, and wherein the first scoring element is positioned between the outer surface of the balloon and the target region in the expanded configuration.

13. A lithotripsy medical device system, comprising:
a console;
an outer shaft having a distal end region and a lumen;
an inner shaft extending within at least a portion of the lumen of the outer shaft, the inner shaft having a distal end region;
a balloon having a proximal end attached to the distal end region of the outer shaft, a distal end attached to the distal end region of the inner shaft, and a plurality of inflation lobes positioned between the proximal end region and the distal end region;
a force generator coupled to the inner shaft and the console; and
a scoring element having a first end attached to the distal end region of the outer shaft and a second end attached to the distal end region of the inner shaft;
wherein at least a portion of the scoring element is free from contact with an outer surface of the balloon.

14. The lithotripsy system of claim 13, wherein the force generator is configured to generate a force wave within a portion of the balloon.

15. The lithotripsy system of claim 14, wherein the force wave is configured to transmit mechanical energy to a target region.

16. The lithotripsy system of claim 15, wherein the first scoring element includes a first scoring wire, and wherein the force wave is configured to vibrate the scoring wire, and wherein the vibration of the scoring wire scores the target region.

17. The lithotripsy system of claim 16, wherein the mechanical energy from the force wave contacts the target region over a time period, and wherein the scoring wire scores the target region over the time period during which the force wave contacts the target region.

18. The lithotripsy system of claim 17, wherein the force wave is transmitted at a frequency between 70 kilohertz and 100 kilohertz.

19. The lithotripsy system of claim 18, wherein the balloon is designed to shift between an unexpanded configuration and an expanded configuration wherein the balloon is in contact with the target region, and wherein the scoring wire is positioned between the outer surface of the balloon and the target region in the expanded configuration.

20. A method for treating a body vessel, the method comprising:
disposing a medical device within the body vessel at a position adjacent to a lesion, the medical device comprising:
an outer shaft having a distal end region and a lumen;
an inner shaft extending within at least a portion of the lumen of the outer shaft, the inner shaft having a distal end region;
a balloon having a proximal end attached to the distal end region of the outer shaft, a distal end attached to the distal end region of the inner shaft, and a plurality of inflation lobes positioned between the proximal end region and the distal end region;
a force generator coupled to the inner shaft; and
a scoring element having a first end attached to the distal end region of the outer shaft and a second end attached to the distal end region of the inner shaft, wherein at least a portion of the scoring element is free from contact with an outer surface of the balloon; and
generating a force wave with the force generator over a time period;
vibrating the scoring wire over the time period, and wherein the force wave and the scoring element at least partially break apart the lesion.
